(19) United States
(12) Patent Application Publication
(10) Pub. No.: US 2010/0305183 A1
(43) Pub. Date: Dec. 2, 2010

(54) METHOD AND COMPOSITION FOR REDUCING REPERFUSION INJURY

(75) Inventors: Alan John Nimmo, Sydney (AU); Karen Margaret Whitfield, Mt. Crosby (AU); Renée Jade Turner, Underdale (AU); Robert Vink, Unley (AU)

Correspondence Address: KLARQUIST SPARKMAN, LLP, 121 SW SALMON STREET, SUITE 1600, PORTLAND, OR, 97204, US

(73) Assignees: Adelaide Research & Innovation Pty Ltd; James Cook University

(21) Appl. No.: 12/518,083

(22) PCT Filed: Dec. 7, 2007
(86) PCT No.: PCT/AU07/01885

§ 371 Date: Aug. 5, 2010

(30) Foreign Application Priority Data
Dec. 8, 2006 (AU) 2006906859

(51) Int. Cl. A61K 31/405; A61P 7/02; A61P 25/28

(52) U.S. Cl. 514/419

(57) ABSTRACT

The present invention relates to a method of preventing and/or reducing reperfusion injury to a tissue, organ, or organ system in a subject. The method includes administering to the subject an effective amount of a substance P receptor antagonist.

This application claims priority from Australian provisional patent application No. 2006906859 filed on 8 Dec. 2006, the contents of which are to be taken as incorporated herein by this reference.

### FIELD OF THE INVENTION

The present invention relates to a method and a composition for reducing reperfusion injury.

### BACKGROUND OF THE INVENTION

Thrombotic occlusions are responsible for a large number of the deaths seen in the population. Indeed, mycocardial infarction and stroke are the major contributors to the overall death rate.

Stroke in particular represents a major clinical burden, representing the primary cause of adult disability, the second-most important cause of dementia, and the third-leading cause of death.

Ischaemic strokes are initiated by the formation of a blood clot (thrombus) within a cerebral blood vessel, or the blockage of a vessel by an embolus. Emboli (typically blood clots) normally are formed elsewhere in the body and travel to the cerebral circulation where they create a blockage. This blockage of a cerebral blood vessel results in an area of the brain being deprived of its normal blood flow and oxygen supply. If the blood supply is not restored, the neurons in the affected area will die, leading to permanent damage. Accordingly, treatment of stroke relies on the restoration of blood flow to the affected area in order to prevent damage from occurring. This may be achieved by delivering a thrombolytic agent, generally within three hours of the stroke.

However, when blood flow is restored to an area that has been subject to ischaemia, reperfusion injury may occur. Such reperfusion injury may not occur as a result of the restoration of blood flow following use of a thrombolyitc agent, but may also occur naturally as a result of endogenous thrombolytic activity, or as a result of surgical or other intervention, for example by revascularisation or the insertion of vascular stents.

In addition, not only does the restoration of blood flow to an area that has been temporarily deprived of blood flow present a problem, but the direct effects of a thrombolytic agent may also be problematic.

In the brain, one of the most significant factors associated with reperfusion injury is a loss of integrity of the blood-brain barrier. This early disruption of the blood-brain barrier is thought to be a precursor for haemorrhagic transformation and poor clinical outcome. Opening of the blood brain barrier also allows for the infiltration of inflammatory cells into the brain, and a subsequent inflammatory response, as well as the formation of cerebral oedema, both of which represent a major component of the secondary injury process. There is clinical evidence that inflammation of the brain is also responsible, at least in part, for the subsequent development of neurologic deficits. In addition, antigens present in the brain are exposed to the systemic immune system, and this may serve as trigger for autoimmune responses which add to the long-term morbidity associated with stroke.

Unfortunately, the problems seen with reperfusion injury are exacerbated by the use of thrombolytic agents, such as tissue plasminogen activators. For example, plasminogen activators themselves may cause disruption of the blood-brain barrier, and therefore an increase in the incidence and magnitude of the inflammatory response and cerebral oedema associated with reperfusion. The problems associated with reperfusion injury, and the exacerbation by thrombolytic agents, has limited the use of plasminogen activators clinically.

Accordingly, there is a need for adjunctive therapy to minimise the problems associated with the reperfusion therapy, and in particular following treatment with thrombolytic agents. The present invention relates to a method for reducing reperfusion injury.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention arises out of the studies into reperfusion injury occurring in the brain following administration of a thrombolytic agent in a reversible stroke model. In particular, it has been found that the use of a substance P receptor antagonist significantly decreases the injury and inflammatory response occurring as a result of reperfusion following the restoration of blood flow to a region after a clot has formed.

This finding indicates that a substance P receptor antagonist may be used as a therapeutic intervention to ameliorate many of the consequences of reperfusion injury, and in particular ameliorate the consequences of the use of thrombolytic agents in the treatment of thrombo-embolic occlusions.

For example, the therapy may reduce the risk of mortality and prevent or reduce long-term neurological deficits and morbidities that would often occur as a result of an ischaemic stroke.

Accordingly, the present invention provides a method of preventing and/or reducing reperfusion injury to a tissue, organ, or organ system in a subject, the method including administering to the subject an effective amount of a substance P receptor antagonist.

The present invention also provides use of a substance P receptor antagonist in the preparation of a medicament for preventing and/or reducing reperfusion injury.

The present invention also provides use of a substance P receptor antagonist and a thrombolytic agent in the preparation of a medicament for preventing and/or treating a thrombo-embolic occlusion.

The present invention also provides use of a substance P receptor antagonist and a thrombolytic agent in the preparation of a medicament for preventing and/or treating stroke.

The present invention also provides a combination product including the following components:
a substance P receptor antagonist; and
a thrombolytic agent;
   wherein the components are provided in a form for separate administration to a subject, or in a form for co-administration to a subject.

The present invention also provides a pharmaceutical composition including an effective amount of a substance P receptor antagonist and a thrombolytic agent.

The present invention also provides a method of preventing and/or reducing oedema to a tissue, organ or organ system in a subject due to reperfusion of the tissue, organ or organ system, the method including administering to the subject an effective amount of a substance P receptor antagonist.

The present invention also provides use of a substance P receptor antagonist in the preparation of a medicament for preventing and/or reducing oedema due to reperfusion of a tissue, organ or organ system.

The present invention also provides a method of preventing and/or reducing an inflammatory response in a tissue, organ or organ system in a subject due to reperfusion of the tissue, organ or organ system, the method including delivering to the tissue, organ or organ system an effective amount of a substance P receptor antagonist.

The present invention also provides use of a substance P receptor antagonist in the preparation of a medicament for preventing and/or reducing an inflammatory response in a tissue, organ or organ system in a subject due to reperfusion of the tissue, organ or organ system.

The present invention also provides a method of treating a thrombo-embolic occlusion in a subject, the method including administering to the subject an effective amount of a substance P receptor antagonist and a thrombolytic agent.

The present invention also provides a method of treating a thrombo-embolic stroke in a subject, the method including administering to the subject an effective amount of a substance P receptor antagonist and a thrombolytic agent.

The present invention also provides use of a substance P receptor antagonist and a thrombolytic agent in the preparation of a medicament for treating a thrombo-embolic stroke.

The present invention also provides a method of improving the prognosis or outcome of a subject suffering from a thrombo-embolic occlusion, the method including administering to the subject an effective amount of a substance P receptor antagonist and a thrombolytic agent.

Various terms that will be used throughout the specification have meanings that will be well understood by a skilled addressee. However, for case of reference, some of these terms will now be defined.

The term “reperfusion injury” as used throughout the specification is to be understood to mean damage to all or part of a tissue, organ or organ system caused when blood supply returns to the tissue, organ or organ system after a period of ischemia.

In this regard, it will be appreciated that an improvement in blood flow to a tissue, organ or organ system may be effected by one or more of the delivery of a thrombolytic agent to an occlusion, a natural process that aids in the restoration of blood flow, and a surgical or other intervention that improves blood flow, such as the use of re-vascularization techniques or the insertion of stents etc.

The term “substance P receptor antagonist” as used throughout the specification is to be understood to mean an agent that directly or indirectly inhibits the binding of substance P to one of its receptors. It will be also appreciated that the substance P receptor antagonist includes a derivative, a variant, an analogue, a pharmaceutically acceptable salt, tautomer or pro-drug of a substance P receptor antagonist.

In this regard, substance P is an excitatory neurotransmitter and is a peptide having the structure RPKPEEFFGLM-NH₂. Methods for determining the ability of an agent to act as a substance P receptor antagonist are known in the art.

The term “thrombolytic agent” as used throughout the specification is to be understood to mean an agent that acts directly or indirectly to reduce any one or more of the size, integrity or permeability of a blood clot, including a thrombotic clot or an embolic clot.

The term “subject” as used throughout the specification is to be understood to mean a human or animal subject.

In this regard, it will be understood that the present invention includes within its scope veterinary applications. For example, the animal subject may be a mammal, a primate, a livestock animal (eg. a horse, a cow, a sheep, a pig, or a goat), a companion animal (eg. a dog, a cat), a laboratory test animal (eg. a mouse, a rat, a guinea pig, a bird, a rabbit), an animal of veterinary significance, or an animal of economic significance.

The term “variant” as used throughout the specification is to be understood to mean an amino acid sequence of a polypeptide or protein that is altered by one or more amino acids. The variant may have “conservative” changes, wherein a substituted amino acid has similar structural or chemical properties to the replaced amino acid (e.g., replacement of leucine with isoleucine). A variant may also have “non-conservative” changes (e.g., replacement of a glycine with a tryptophan) or a deletion and/or insertion of one or more amino acids. The term also includes within its scope any insertions/deletions of amino acids for a particular polypeptide or protein. A “functional variant” will be understood to mean a variant that retains the functional capacity of a reference protein or polypeptide.

Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Under some circumstances, substitutions within the aliphatic group alanine, valine, leucine and isoleucine are also considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Substitutions as described above are contemplated within the scope of the present invention.

The term “treat” as used throughout the specification is to be understood to mean an intervention that improves the prognosis and/or state of a subject.

### BRIEF DESCRIPTION OF THE FIGURES

FIG. 1 shows the extent of Evan's blue leakage into brain tissue following injury with or without the administration of N-acetyl-L-tryptophan.

FIG. 2 shows the permeability of the blood brain induced by tPA alone or in conjuction with N-acetyl-L-tryptophan.

FIG. 3 shows the extent of oedema in a reversible stroke model with or without the administration of N-acetyl-L-tryptophan.

FIG. 4 shows the improvement in motor function in an animal subject to ischaemia/reperfusion injurt with or without administration of N-acetyl-L-tryptophan or L733,060.

FIG. 5 shows the level of the mRNAs for various inflammatory mediators in the brain following reperfusion and hypotension with or without the administration of N-acetyl-L-tryptophan.

FIG. 6 shows the serum levels of IL-6 following reperfusion and hypotension with or without the administration of N-acetyl-L-tryptophan.

### GENERAL DESCRIPTION OF THE INVENTION

As described above, in one embodiment the present invention provides a method of preventing and/or reducing reperfusion injury to a tissue, organ or organ system, the method including delivering to the tissue, organ or organ system an effective amount of one or more substance P receptor antagonists.

This embodiment of the present invention is directed to preventing and/or reducing reperfusion injury to a tissue, organ or organ system under circumstances where blood flow is improved to the tissue, organ or organ system. Blood flow to the tissue, organ or organ system may be partially or fully occluded. This embodiment of the present invention may be used, for example, to prevent and/or treat a reperfusion injury in a subject.

Methods of delivery of one or more substance P receptor antagonists to a tissue, organ or organ system are as described in detail herein. In one embodiment, the substance P receptor antagonist is delivered to the tissue, organ or organ system by administration to the subject.

Accordingly, in another embodiment the present invention provides a method of preventing and/or reducing reperfusion injury to a tissue, organ, or organ system in a subject, the method including administering to the subject an effective amount of a substance P receptor antagonist.

It will be appreciated in this regard that an improvement in blood flow to an occlusion may be brought about by one or more of the administration of a thrombolytic agent, natural thrombolytic processes, and surgical or other interventions, such as re-vascularisation therapy and the use of stents etc.

In one embodiment, the reperfusion injury to the tissue, organ or organ system is due. use of one or more thrombolytic agents to improve blood flow to the tissue, organ or organ system. This embodiment of the present invention may be used, for example, to prevent and/or treat reperfusion injury in a subject following the administration of a thrombolytic agent.

Accordingly, in another embodiment the present invention provides a method of preventing and/or reducing reperfusion injury to a tissue, organ, or organ system in a subject due to administration of a thrombolytic agent to the subject, the method including administering to the subject an effective amount of a substance P receptor antagonist.

The thrombolytic agent in the various embodiments of the present invention is an agent that acts directly or indirectly to reduce any one or more of the size, integrity or permeability of a blood clot. Identification of an agent as a thrombolytic agent may be determined by a method known in the art. Thrombolytic agents are described for example in “New Therapeutic Agents in Thrombosis and Thrombolysis” (2003) Edited By Joseph Loscalzo and Arthur Asao Sasahara, Informa Health Care.

Examples of thrombolytic agents in the various embodiments of the present invention include tissue plasminogen activators (eg tPA), urokinase, streptokinase, or variants, pro-peptide forms or functionally equivalent analogues of any of these agents.

For example, tPA is a serine protease that is produced from a variety of sources and which is readily commercially available. A number of variants of tPA exist that may be used as thrombolytic agents. For example, variants of lysine 227 substituted with other amino acids may be used. Other variants have a deletion of from 3 to 25 amino acids from the C-terminus and/or have a substitution of at least one of asparagine 117, asparagine 184 and asparagine 448 with another amino acid.

The appropriate thrombolytic agent(s) for use in the various embodiments of the present invention will depend upon the particular clinical indication and the characteristics of the subject, and as such may be selected by an appropriate practioner. Commercially available thrombolytic agents include reteplase, alteplase, tenecteplase, urokinase, prourokinase, anisoylated purified streptokinase activator complex, and streptokinase.

Alteplase (A ctivase) is a recombinant tissue plasminogen activator which is identical to native tissue plasminogen activator. This thrombolytic agent is often used for the treatment of coronary artery thrombosis, pulmonary embolism, and acute ischemic stroke.

Reteplase is a recombinant tissue plasminogen activator containing 355 of the 527 amino acids of the native protein, and which has a faster plasma clearance and shorter half-life than alteplase. The shorter half-life makes the drug useful for double-bolus dosing, as compared to alteplase, which is given by a bolus followed by an intravenous infusion.

Urokinase is most often used for peripheral intravascular thrombus. Prourokinase is a relatively inactive precursor that must be converted to urokinase before it becomes active in vivo.

Streptokinase binds with free circulating plasminogen (or with plasmin) to form a complex that can convert additional plasminogen to plasmin. Anisoylated purified streptokinase activator complex is a complex of streptokinase and plasminogen that does not require free circulating plasminogen to be effective.

In one embodiment, the thrombolytic agent is a tissue plasminogen activator, including a functional variant or analogue of a native tissue plaminogen activator.

As described previously herein, the substance P receptor antagonist in the various embodiments of the present invention is an agent that directly or indirectly inhibits the binding of substance P to one of its receptors. It will be also appreciated that the substance P receptor antagonist includes a derivative, a variant, an analogue, a pharmaceutically acceptable salt, tautomer or pro-drug of a substance P receptor antagonist.

In this regard, substance P is an excitatory neurotransmitter and is peptide a having the structure RPKPEEFFGLM-NH₂. Substance P binds to a number of receptors including the NK1 receptor (neurokinin 1 receptor), the NK2 receptor and the NK3 receptor. Substance P antagonists inhibit the binding of substance P to any one of its receptors. It will be appreciated that the term “substance P” includes within its scope various variants, truncated forms or analogues of the peptide, for example as described in U.S. Pat. No. 4,481,139.

The identification of a substances as a substance P receptor antagonist may be determined by a method known in the art, for example as described in U.S. Pat. Nos. 5,990,125, 6,482,829; and 5,972,938; and US patent application 20030083345.

Examples of substance P receptor antagonists are shown in Tables 1 to 3.

TABLE 1
NK1 Receptor Antagonists

| Chemical | |
|---|---|
| Code | Chemical Name |
| CGP49823 | (2R,4S)-2-benzyl-1-(3,5-dimethylbenzoyl)-N-[(4-quinolinyl)methyl]-4-piperineamine) dihydrochloride |
| CP-96,345 | 2S,3S)-cis-(2(diphenylmethyl)-N-[(2-methoxyphenyl)methyl]-1-azabicyclo[2.2.2]octan-3-amine |
| CP-99,994 | ((2S,3S)-cis-3-(2-methoxybenzylamino)-2-phenyl-piperidine)dihydrochloride |
| CP-122,721 | (+)-2S,3S)-3-(2-methoxy-5-trifluoromethoxybenzyl)amino-2-phenylpiperidine |
| FK 888 | (N2-[(4R)-4-hydroxy-1-(1-methyl-III-indol-3-yl)carbonyl-L-propyl\-N-methyl-N-phenylmethyl-L-3-(2-naphthyl)-alaninamide |
| GR203040 | (2S,3S and 2R,3R)-2-methoxy-5-tetrazol-1-yl-benzyl-(2-phenyl-piperidin-3-yl)-amine |
| GR-205171 | 3-Piperidinamine,N-[[2-methoxy-5-[5-(trifluoromethyl)-1H-tetrazol-1yl]phenyl]methyl]-2-phenyl-, (2S-cis)- |
| GR 82334 | [D-Pro9,)spiro-gamma-lactam]Leu10, Trp11]physalaemin-(1-11) |
| GR 94800 | PhCO-Ala-Ala-DTrp-Phe-DPro-Pro-Nle-NH2 |
| HSP-117 | 3-Piperidinamine, N-[[2,3-dihydro-5-(1-methylethyl)-7-benzofuranyl]methyl)2-phenyl-, dihydrochloride, (2S-cis)- |
| L 703,606 | 1-Azabicycio[2.2.]octan-3-amine, 2-(diphenylmethyl)-N-[(2-idophenyl)methyl]-, (2S-cis)-, oxalate |
| L 732,138 | N-acetyl-L-tryptophan |
| L 733,060 | ((2S,S)-3-((3,5-bis(trifluoromethyl)phenyl)methyloxy)-2-phenyl piperidine |
| L 742,694 | (2-(S)-(3,5-bis(trifluromethyl)benzyloxy)-3-(S)-phenyl-4-(5-(3-oxo-1,2,4-triazolo)methylmorpholine |
| L 754,030 | 2-(R)-(1-(R)-3,5-bis(trifluoromethyl)phenylethoxy)-3-(S)-(4-fluoro)phenyl-4-(3-oxo-1,2,4-triazol-5-yl)methylmorpholine |
| L668,169 | L-Phenylalanine, N-[2-[3-[[N-[2-(3-[(N-[2-[3-amino-2-oxo-1-pyrrolidinyl)-4-methyl-1-oxopentlyl]-L-methionyl-L-glutaminyl- |
| | D-tryplophyl-N-methyl-L-phenylalanyl]amino]-2-oxo-1-pyrrolidinyl]-4-methyl-1-oxopentyl]-L-methionyl-L-glutaminyl-D- |
| | tryptophyl-N-methyl-,cyclic (8->1)-peptide, [3R-[1[S*[R*(S*)]], 3R*]]- |
| LY 303241 | 1-Piperazineacetamide, N-[2-[acetyl[(2-methoxyphenyl)methyl]amino]-1-(1H-indol-3-ylmethyl)(ethyl]-4-phenyl-, (R)- |
| LY 303870 | (R)-1-[N-(2-methoxybenzyl)acetylamino]-3-(1H-indol-3-yl)-2-[N-(2-(4-(piperidinyl)piperidin-1-yl)acetyl)amino]propane |
| LY 306740 | 1-Piperazineacetamide, N-[2-′acetyl[(2-methoxypehenyl)methyl]amino]-1-(1H-indol-3-ylmethyl)ethyl]-4-cyclohexyl-, (R)- |
| MEN 11149 | 2-(2-naphthyl)-1-N[(1R,2S)-2-N-[1(H)indol-3-ylcarbonyl]aminocyclohexanecarbonyl]-1-[N′-ethyl-N′- |
| | (4-methylphenylacetyl)]diaminoethane |
| MK-869 | 3H-1,2,4-Triazol-3-one, 5-[[2-[1-[3,5-bis(trifuoromethyl)phenyl]ethoxy]-3-(4-fluorophenyl)-4-morpholinyl]methyl]-1,2- |
| | dihydro-,[2R-[2a(R*), 3a]]- |
| PD 154075 | (2-benzofuran)-CH2OCO]—(R)-alpha-MeTrp-(S)—NHCH(CH3)Ph |
| R-544 | Ac-Thr-D-Trp(FOR)-Phe-N-MeBzl |
| RP-67580 | (3aR,7aR)-7,7-diphenyl-2[1-imino-2(2-methoxyphenyl)-(ethyl]+++perhydroisoindol-4-one hydrochloride |
| RPR 100893 | (3aS,4S,7aS)-7,7-diphenyl-4-(2-methoxyphenyl)-2-[(S)-2-(2-methoxyphenyl)proprionyl]perhydroisoindol-4-ol |
| Spendide | Tyr-D-Phe-Phe-D-Ilis-Leu-Met-NII2 |
| Spantide II | D-NicLys1, 3-Pal3, D-Cl2Phe5, Asn6, D-Trp7.0, Nlel1-substance P |
| Spantide III | L-Norlcucinamide, N6-(3-pyridinylcarbonyl)-D-lysyl-L-prolyl-3-(3-pyridinyl)-L-alanyl-L-prolyl-3,4-dichioro-D-phenylalanyl- |
| | L-asparaginyl-D-tryptophyl-L-phenylalanyl-3-(3-pyridinyl)-D-alanyl-L-leucyl- |
| SR140333 | (S)-1-[2-[3-(3,4-dichlorphenyl)-1 (3-isopropoxyphenylacetyl) piperidin-3-yl] ethyl]-4-phenyl-1 azaniabicyclo [2.2.2]octane |
| WIN-41,708 | (17beta-hydroxy-17alpha-ethynyl-5alpha-androstano[3,2-b]pyrimido[1,2-a]benzimidazole |
| WIN-62,577 | 1H-Benzimidazo[2,1-b]cyclopenta[5,6]naphtha[1,2-g]quinazolin-1-ol, 1-ethynyl-2,3,3a,3b,4,5,15,15a,15b,16,17,17a- |
| | dodcachydro-15a, 17a-dimethyl-, (1R,3aS,3bR,15aR,15bS,17aS)- |

TABLE 2
NK2 Receptor Antagonists

| Chemical | |
|---|---|
| Code | Chemical Name |
| SR-48,968 | (S)—N-methyl-N[4-(4-acetylamino-4- |
| | [phenylpiperidino)-2-(3,4-dichlorophenyl)-butyl]benzamide |
| L-659,877 | Cyclo[Gin,Trp,Phe,Gly,Leu,Met] |
| MEN 10627 | Cyclo(Met-Asp-Trp-Phe-Dap-Leu)cyclo(2beta-5beta) |
| SR 144190 | (R)-3-(1-[2-(4-benzoyl-2-(3,4-difluorophenyl)-morpholin- |
| | 2-yl)-ethyl]-4-phenylpiperidin-4-yl)-1-dimethylurea |
| GR 94800 | PhCO-Ala-Ala-D-Trp-Phe-D-Pro-Pro-Nle-NH2 |

TABLE 3
NK3 Receptor Antagonists

| Chemical | |
|---|---|
| Code | Chemical Name |
| SR-142,801 | (S)—(N)-(1-[3-(1-benzoyl-3-(3,4-dichlorophenyl)piperidin- |
| | 3-yl)propyl)-4-phenylpiperidin-4-yl)-N-methyl acetaide |
| R820 | 3-Indolylcarbonyl-Ilyp-Phg-N(Me)-Bzl |
| R486 | H-Asp-Ser-Phe-Trp-beta-Ala-Leu-Met-NH2 |
| SB 222200 | (S)-(−)-N-(a-ethylbenzyl)-3-methyl-2-phenylquinoline- |
| | 4-carboximide |
| I, 758,298 | Phosphonic acid, [3-[[2-[1-[3,5- |
| | bis(trfluoromethyl)phenyl]ethoxy]-3-(4-fluorophenyl)- |
| | 4-morpholinyl]methyl]-2,5-dihydro-4oxo- |
| | 1H-1,2,4-triazol-1-yl]-, [2R-[2a(R*), 3a]]- |
| NK-608 | (2R,4S)-N-[1-[3,5-bis(trifluormethyl)-benzoyl)-2- |
| | (4-chloro-benzyl)-4-piperidinyl]-quinoline-4-carboxamide |

Other examples of substance P receptor antagonists are as described in U.S. Pat. Nos. 4,481,139 and 5,977,104. Examples of NK1 receptor antagonists are as described in U.S. Pat. No. 5,990,125.

Tachykinin antagonists (as described in U.S. Pat. No. 4,981,744) may also be used as substance P antagonists. Other examples of substance P receptor antagonists include piperdine and morpholine derivatives (as described in U.S. Pat. No. 4,985,896), piperazino (as described in U.S. Pat. No. 5,981,52), piperidinyl compounds as NK1 or NK2 antagonists (as described in U.S. Pat. No. 5,998,444), N-benzyl-4-tolylnicotin-amides and related compounds as NK1 receptor antagonists (as described in European patent application EP-A-1035115), phenyl and pyridinyl derivatives as NK1 receptor antagonists (as described in international patent application WO 0050398), and 3-phenylpyridines, biphenyl derivatives, 5-phenyl-pyrimidine derivatives and 4-phenyl-pyrimidine derivatives (as described in international patent applications WO 0050401, WO 0053572, WO 0073278 and WO 0073279).

In one embodiment, the substance P receptor antagonist is one or more of a NK1 receptor antagonist, a NK2 receptor antagonist, and a NK3 receptor antagonist.

In one embodiment, the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138 (N-acetyl-L-typtophan), L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577, or a derivative, a variant, an analogue, a pharmaceutically acceptable salt, tautomer or pro-drug thereof.

In another embodiment, the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, tautomer or pro-drug thereof.

In another embodiment, the NK3 receptor antagonist is selected from one or more of the group consisting of SR-143,801, R820, R486, SB222200, L758,298 and NKP608, or a derivative, a variant, an analogue, a pharmaceutically acceptable salt, tautomer or pro-drug thereof.

In one embodiment, the substance P receptor antagonist is L-732,138, namely N-acetyl-L-tryptophan, or a derivative, analogue, pharmaceutically acceptable salt, tautomer or pro-drug thereof. Examples include lipid soluble analogues, N-acetyl-L-tryptophan 3,5-bis(trifluoromethyl)benzyl ester and N-acetyl tryptophan methyl ester.

In another embodiment, the substance P receptor antagonist is L-733060, or a derivative, analogue, pharmaceutically acceptable salt, tautomer or pro-drug thereof.

Generally, any tissue or organ may be susceptible to reperfusion injury. Specific examples of tissues, organs or organ systems that may be affected by reperfusion injury in the various embodiments of the present invention include brain, the central nervous system, lung, heart and kidney.

In one embodiment, the organ or tissue is the brain.

In one embodiment, the reperfusion injury of the tissue, organ or organ system is due to one or more of odcoma, inflammation or oxidative damage of the tissue, organ or organ system.

In the case of reperfusion of the brain, given that the brain is normally shielded from the immune system, the reperfusion injury may also be an autoimmune response to one or more antigens present in the brain.

In one embodiment, the reperfusion injury in the brain is due to reperfusion of all or part of the brain following a stroke.

The present invention may also be used in the preparation of a medicament for preventing and/or reducing reperfusion injury.

Accordingly, in another form the present invention provides use of a substance P receptor antagonist in the preparation of a medicament for preventing and/or reducing reperfusion injury.

In one embodiment, the substance P receptor antagonist may be used in the preparation of a medicament for preventing and/or reducing reperfusion injury due to administration of a thrombolytic agent, for example to prevent and/or treat a clot affecting a tissue, organ or organ system.

Accordingly, in another embodiment the present invention provides use of a substance P receptor antagonist in the preparation of a medicament for preventing and/or reducing reperfusion injury due to administration of a thrombolytic agent.

The present invention may also be used in the preparation of a medicament for preventing and/or treating a thrombo-embolic occlusion.

In another embodiment, the present invention provides use of a substance P receptor antagonist and a thrombolytic agent in the preparation of a medicament for preventing and/or treating a thrombo-embolic occlusion.

The present invention may also be used in the preparation of a medicament for preventing and/or treating a stroke arising from a thrombo-embolic stroke.

Accordingly, in another embodiment the present invention provides use of a substance P receptor antagonist and a thrombolytic agent in the preparation of a medicament for preventing and/or treating stroke.

Methods for the preparation of medicaments are known in the art, for example as described in Remington's Pharmaceutical Sciences, 18th ed., 1990, Mack Publishing Co., Easton, Pa. and U.S. Pharmacopeia: National Formulary, 1984, Mack Publishing Company, Easton, Pa.

As described previously herein, the delivery of the substance P receptor antagonist to the tissue, organ or organ system includes administration of the substance P receptor antagonist to a subject.

Accordingly, in another embodiment the present invention provides a method of preventing and/or reducing reperfusion injury to an organ or tissue in a subject, the method including administering to the subject an effective amount of a substance P receptor antagonist.

The present invention may also be used to prevent and/or reduce oedema to an organ, organ system or tissue due to reperfusion of the tissue, organ or organ system.

Accordingly, in another form the present invention provides a method of preventing and/or reducing oedema to a tissue, organ or organ system in a subject due to reperfusion of the tissue, organ or organ system, the method including administering to the subject an effective amount of a substance P receptor antagonist.

In one embodiment, the reperfusion is due to delivery of a thrombolytic agent to improve blood flow to the tissue, organ or organ system.

Accordingly, in another embodiment the present invention provides a method of preventing and/or reducing oedema to a tissue, organ or organ system in a subject due to reperfusion following administration of a thrombolytic agent, the method including delivering to the tissue, organ or organ system an effective amount of a substance P receptor antagonist.

As described previously herein, in one embodiment the delivery of the substance P receptor antagonist is by way of administration to the subject.

Generally, any tissue, organ or organ system is susceptible to oedema. Specific examples of tissues, organs or organ systems that are susceptible to oedema include brain, lung, heart, kidney and liver.

In one embodiment, the oedoma occurs in the brain (ie a cerebral oedema), for example as occurs following administration of a thrombolytic agent to a subject to treat a stroke.

The present invention may also be used in the preparation of a medicament for preventing and/or reducing oedema due to reperfusion of the tissue, organ or organ system.

Accordingly, in another embodiment the present invention provides use of a substance P receptor antagonist in the preparation of a medicament for preventing and/or reducing oedema due to reperfusion of a tissue, organ or organ system.

In one embodiment, the reperfusion is due to the administration of a thrombolytic agent, to improve blood flow to the tissue, organ or organ system.

Accordingly, in another embodiment the present invention provides use of a substance P receptor antagonist in the preparation of a medicament for preventing and/or reducing oedema due to reperfusion of a tissue, organ or organ system following administration of a thrombolytic agent.

The present invention may also be used to prevent and/or reduce an inflammatory response in a tissue, organ or organ system due to reperfusion of a tissue, organ or organ system.

Accordingly, in another embodiment the present invention provides a method of preventing and/or reducing an inflammatory response in a tissue, organ or organ system due to reperfusion of the tissue, organ or organ system, the method including delivering to the tissue, organ or organ system an effective amount of a substance P receptor antagonist.

In one embodiment, the reperfusion is due to administration of a thrombolytic agent, to improve blood flow to the tissue, organ or organ system.

Accordingly, in another embodiment the present invention provides a method of preventing and/or reducing an inflammatory response in a tissue, organ or organ system due to use of a thrombolytic agent to improve blood flow to an occluded tissue, organ or organ system, the method including delivering to the tissue, organ or organ system an effective amount of a substance P receptor antagonist.

Generally, any tissue, organ or organ system is susceptible to inflammation. Specific examples of tissues, organs or organ systems that are susceptible to an inflammatory response include brain, central nervous system, lung, heart, kidney and liver.

In one embodiment, the inflammatory response occurs in the brain. In a specific embodiment, the inflammatory response is due to administration of a thrombolytic agent to a subject to treat a stroke.

The present invention also may be used in the preparation of a medicament for preventing and/or reducing an inflammatory response in a tissue, organ or organ system due to reperfusion of the tissue, organ or organ system.

Accordingly, in another embodiment the present invention provides use of a substance P receptor antagonist in the preparation of a medicament for preventing and/or reducing an inflammatory response due to reperfusion of the tissue, organ or organ system.

In one embodiment, the reperfusion is due to administration of a thrombolytic agent to improve blod flow to the tissue, organ or organ system.

Accordingly, in another embodiment the present invention provides use of a substance P receptor antagonist in the preparation of a medicament for preventing and/or reducing an inflammatory response in a tissue, organ or organ system resulting from administration of a thrombolytic agent.

The present invention also provides an improved therapy to treat a thrombo-embolic occlusion in a subject.

Accordingly, in another embodiment the present invention provides a method of treating a thrombo-embolic occlusion in a subject, the method including administering to the subject an effective amount of a substance P receptor antagonist and a thrombolytic agent.

In one embodiment, the thrombo-embolic occlusion is a stroke, a pulmonary occlusion, or a coronary artery occlusion.

In one specific embodiment, the thrombo-embolic occlusion is a stroke.

Accordingly, in another embodiment the present invention provides a method of treating a thrombo-embolic stroke in a subject, the method including administering to the subject an effective amount of a substance P receptor antagonist and a thrombolytic agent.

The present invention also may be used in the preparation of a medicament for treating stroke.

Accordingly, in another embodiment the present invention provides use of a substance P receptor antagonist and a thrombolytic agent in the preparation of a medicament for treating a thrombo-embolic stroke.

The present invention may also be used to improve the prognosis or outcome after a subject suffers a thrombo-embolic occlusion.

Accordingly, in another embodiment the present invention provides a method of improving the prognosis or outcome of a subject suffering from a thrombo-embolic occlusion, the method including administering to the subject an effective amount of a substance P receptor antagonist and a thrombolytic agent.

In the case where the thrombo-embolic occlusion results in a stroke, in one embodiment the improvement in prognosis or outcome is an improvement in the motor and/or cognitive prognosis or outcome.

The delivery of the substance P receptor antagonist and the thrombolytic agent to the tissue, organ or organ system in the various embodiments of the present invention may include administration of the substance P receptor antagonist and the thrombolytic agent to a subject.

In this regard, it will also be appreciated that the thrombolytic agent and the substance P receptor antagonist in the various embodiments of the present invention may be delivered to the tissue, organ or organ system simultaneously, or may be delivered sequentially.

In the case of administration to a subject, the two agents may be separately administered to the subject in a suitable form, or alternatively, be co-administered to the subject in a suitable form.

In this regard, co-administration generally means that the actives are present in the subject during a specified time interval. Typically, if a second agent is administered within the half-life of the first agent, the two agents are considered co-administered.

Thus, the present invention may also be used for a combination product including a substance P receptor antagonist and a thrombolytic agent.

Accordingly, in another embodiment the present invention provides a combination product including the following components:
a substance P receptor antagonist; and
a thrombolytic agent;
   wherein the components arc provides in a form for separate administration to a subject, or in a form for co-administration to a subject.

The subject may be suffering from, or susceptible to, one or more of the various indications as described herein.

For example, the subject for treatment with the combination product may be suffering from, or susceptible to, a thrombo-embolic occlusion, such as a stroke.

Accordingly, in another embodiment the present invention provides a combination product including the following components:
a substance P receptor antagonist; and
a thrombolytic agent;
   wherein the components are provides in a form for separate administration to a subject, or in a form for co-administration to a subject, wherein the subject is suffering from, or susceptible to, a thrombo-embolic occlusion.

The components of the combination product may packaged separately or together in suitably sterilized containers such as ampoules, bottles, or vials, either in multi-dose or in unit dosage forms. The containers are typically hermetically sealed. Methods are known in the art for the packaging of the components.

In one embodiment, the components are provided in an isotonic saline solution.

The delivery of the substance P receptor antagonist and/or the thrombolytic agent to the tissue, organ or organ system in various embodiments of the present invention may include the delivery of a pharmaceutical composition including the substance P receptor antagonist and the thrombolytic agent. In this case, the substance P receptor antagonist and the thrombolytic agent may be prepared into a suitable pharmaceutical composition.

Accordingly, in another embodiment the present invention provides a pharmaceutical composition including an effective amount of a substance P receptor antagonist and a thrombolytic agent.

A suitable dosage of the substance P receptor antagonist for delivery to the desired site of action may be selected. Generally, the dosage of the substance P receptor antagonist to a subject in the various embodiments of the present is in the range from 0.1 mg/kg to 100 mg/kg. Typically, the dosage is in the range from 0.25 mg/kg to 25 mg/kg.

For example, in the case of administration of the substance P receptor antagonist to a subject, the dosage of the substance P receptor antagonist in the pharmaceutical composition may be 10-5,000 mg per subject, and generally will be in the range of 50-2,000 mg per subject.

In the case of a substance P receptor antagonist, suitable dosages are as described in U.S. Pat. No. 4,990,125 and U.S. Pat. No. 5,977,104. Examples of formulations are described in U.S. Pat. No. 5,990,125.

A suitable dosage of the thrombolytic agent for delivery to the desired site of action may also be selected, depending upon the particular occlusion and other factors specific to the particular subject. Generally, the dosage of the thrombolytic agent administered to a subject in the various embodiments of the present is in the range from 0.1 mg/kg to 100 mg/kg. Typically, the dosage is in the range from 0.25 mg/kg to 25 mg/kg.

For example, in the case of tPA (Alteplase) for use in ischemic stroke, a dose of 0.9 mg/kg intravenously over one hour may be suitable.

In the case of the use of a thrombolytic agent for the treatment of myocardial infarction, doses of the following agents may be suitable:

Streptokinase: 1.5×10³ IU for one hour;

tPA (Alteplase): a loading dose of 15 mg, followed by a dose of 50 mg over 30 minutes and 35 mg over the following hour;

tPA (Reteplase): a dose of 10+10 U double bolus, 10 U bolus over 2 min, wait 30 minutes and repeat 10 U over 2 min;

tPA (Tenecteplase): a dose of 30-50 mg by single bolus body weight.

In the case of the use of a thrombolytic agent for the treatment of pulmonary embolism, doses of the following agents may be suitable:

Streptokinase: a loading dose of 250,000 IU over 30 min, followed by a maintenance dose of 100,000 IU/hr for 24 hours;

tPA (Alteplase): a maintenance dose of 100 mg over 2 hours;

Urokinase: a loading dose of 2,000 IU/lb over 10 minutes followed by a maintenance dose of 2,000 IU/lb/hr for 12 hours.

As described previously herein, methods for the preparation of pharmaceutical compositions are known in the art, for example as described in Remington's Pharmaceutical Sciences, 18th ed., 1990, Mack Publishing Co., Easton, Pa. and U.S. Pharmacopeia: National Formulary, 1984, Mack Publishing Company, Easton, Pa.

For example, a composition for intravenous administration may include 1 mg/ml of the thrombolytic agent (eg tPA) and 0.3-4 mg/ml of the substance P receptor antagonist in isotonic saline.

As discussed previously herein, the substance P receptor antagonist and the thrombolytic agent may be delivered directly or indirectly. For example, direct delivery may be achieved by injection into the affected organ or tissue.

Alternatively, in the various embodiments of the present invention the substance P receptor antagonist and the thrombolytic agent may be delivered to an organ or tissue in a subject by way of administration to the subject.

The thrombolytic agent and/or the substance P receptor antagonist may be delivered in a form and at a concentration suitable to allow the agents to reach the desired site of action and have the effect, as previously discussed herein.

The administration of the thrombolytic agent and/or the substance P receptor antagonist in the various embodiments of the present invention may utilise a suitable administration regime to produce the desired effect.

For example, generally the substance P receptor antagonist will be administered to a subject concurrently with or shortly after administration of the thrombolytic agent. In this regard, the administration of the thrombolytic agent is generally within 3 hours of a thrombotic occlusion occurring. For example, administration by a single, bolus intraveneous injection at a dose of 80 mg may be suitable.

However, it will also be appreciated that prophylactic administration of the substance P receptor antagonist is comtemplated, particularly in subjects susceptible to suffering from a thrombo-embolic occlusion.

The delivery of the thrombolytic agent and/or the substance P receptor antagonist may be by any suitable means, such as administered orally, parenterally, topically, and therefore transit time of the agents must be taken into account.

The thrombolytic agent and/or the substance P receptor antagonist may be formulated into a pharmaceutical composition for administration to a subject, and as such the composition may be packaged in a suitably sterilized container such as an ampoule, bottle, or vial, either in multi-dose or in unit dosage forms. The containers will generally be hermetically sealed. Methods are known in the art for the packaging of components for pharmaceutical administration.

The effective amount of the thrombolytic agent and/or the substance P receptor antagonist to be administered to the subject in the various embodiments of the present invention is not particularly limited, so long as it is within such an amount and in such a form that generally exhibits a useful or therapeutic effect. The term “therapeutically effective amount” is the quantity which, when administered to a subject in need of treatment, improves the prognosis and/or state of the subject. The amount to be administered to a subject will depend on the particular characteristics of the disease, condition or state in the subject, the mode of administration, and the characteristics of the subject, such as general health, other diseases, age, sex, genotype, body weight and tolerance to drugs. A person skilled in the art will be able to determine appropriate dosages depending on these and other factors.

As discussed previously herein, administration and delivery of the compositions according to the invention may be for example by the intravenous, intraperitoneal, subcutaneous, intramuscular, oral, or topical route, or by direct injection. The mode and route of administration in most cases will depend on the type of disease, condition or state being treated.

The dosage form, frequency and will depend on the mode and route of administration.

As described above, the administration of the thrombolytic agent and/or the substance P receptor antagonist may also include the use of one or more pharmaceutically acceptable additives, including pharmaceutically acceptable salts, amino acids, polypeptides, polymers, solvents, buffers, excipients, preservatives and bulking agents, taking into consideration the particular physical, microbiological and chemical characteristics of the agents to be administered.

For example, the thrombolytic agent and/or the substance P receptor antagonist can be prepared into a variety of pharmaceutically acceptable compositions in the form of, e.g., an aqueous solution, an oily preparation, a fatty emulsion, an emulsion, a lyophilised powder for reconstitution, etc. and can be administered as a sterile and pyrogen free intramuscular or subcutaneous injection or as injection to an organ, or as an embedded preparation or as a transmucosal preparation through nasal cavity, rectum, uterus, vagina, lung, etc. The composition may be administered in the form of oral preparations (for example solid preparations such as tablets, caplets, capsules, granules or powders; liquid preparations such as syrup, emulsions, dispersions or suspensions).

Compositions containing the thrombolytic agent and/or the substance P receptor antagonist may also contain one or more pharmaceutically acceptable preservatives, buffering agents, diluents, stabilisers, chelating agents, viscosity enhancing agents, dispersing agents, pH controllers, or isotonic agents.

Examples of suitable preservatives are benzoic acid esters of para-hydroxybenzoic acid, propylene glycol, phenols, phenylethyl alchohol or benzyl alcohol. Examples of suitable buffers are sodium phosphate salts, citric acid, tartaric acid and the like. Examples of suitable stabilisers are, antioxidants such as alpha-tocopherol acetate, alpha-thioglycerin, sodium metabisulphite, ascorbic acid, acetylcysteine, 8-hydroxyquinoline, chelating agents such as disodium edetate. Examples of suitable viscosity enhancing agents, suspending or dispersing agents are substituted cellulose ethers, substituted cellulose esters, polyvinyl alchohol, polyvinylpyrrolidone, polyethylene glcols, carbomer, polyoxypropylene glycols, sorbitan monooleate, sorbitan sesquioleate, polyoxyethylene hydrogenated castor oil 60.

Examples of suitable pH controllers include hydrochloric acid, sodium hydroxide and the like. Examples of suitable isotonic agents are glucose, D-sorbitol or D-mannitol, sodium chloride.

The administration of thrombolytic agent and/or the substance P receptor antagonist in the various embodiments of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, glidant, anti-adherant, binder, flavorant or sweetener, taking into account the physical, chemical and microbiological properties of the agents being administered.

For these purposes, the composition may be administered orally, parenterally, by inhalation spray, adsorption, absorption, topically, rectally, nasally, mucosally, transdermally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, infrasternal, and intracranial injection or infusion techniques.

When administered parenterally, the compositions will normally be in a unit dosage, sterile, pyrogen free injectable form (solution, suspension or emulsion, which may have, been reconstituted prior to use) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable vehicles, dispersing or wetting agents and suspending agents. The sterile injectable forms may also be sterile injectable solutions or suspensions in non-toxic parenterally acceptable diluents or solvents, for example, as solutions in 1,3-butanediol. Among the pharmaceutically acceptable vehicles and solvents that may be employed are water, ethanol, glycerol, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

In addition, the compositions may be in a form to be reconstituted prior to administration. Examples include lyophilisation, spray drying and the like to produce a suitable solid form for reconstitution with a pharmaceutically acceptable solvent prior to administration.

Compositions may include one or more buffers, bulking agents, isotonic agents and cryoprotcctants and lyoprotectants. Examples of excipients include, phosphate salts, citric acid, non-reducing such as sucrose or trehalose, polyhydroxy alcohols, amino acids, methylamines, and lyotropic salts which are usually used instead of reducing sugars such as maltose or lactose.

When administered orally, the thrombolytic agent and/or the substance P receptor antagonist will usually be formulated into unit dosage forms such as tablets, caplets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include excipients such as lactose, dextrose, sucrose, sorbitol, mannitol, starches, gum acacia, calcium phosphate, mineral oil, cocoa butter, oil of theobroma, alginates, tragacanth, gelatin, syrup, substituted cellulose ethers, polyoxyethylene sorbitan monolaurate, methyl hydroxybenzoate, propyl hydroxybenzoate, talc, magnesium stearate, and the like.

A tablet may be made by compressing or molding the agent optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The administration of the thrombolytic agent and/or the substance P receptor antagonist may also utilize controlled release technology.

The thrombolytic agent and/or the substance P receptor antagonist may also be administered as a sustained-release pharmaceutical composition. To further increase the sustained release effect, the agent may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof, carboxymethylcellulose sodium hydroxypropylcellulose ether, collagen polyethylene glycol polyethylene oxide, hydroxypropylmethylcellulosemethylcellulose, polyvinyl alcohol, polyvinylpyrrolidone.

Alternatively, the thrombolytic agent and/or the substance P receptor antagonist may be incorporated into a hydrophobic polymer matrix for controlled release over a period of days. The agent may then be moulded into a solid implant, or externally applied patch, suitable for providing efficacious concentrations of the agents over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers, which may be used externally or internally.

Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time-release characteristics and release kinetics. The agent may then be moulded into a solid implant suitable for providing efficacious concentrations of the agents over a prolonged period of time without the need for frequent re-dosing. The agent can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

For topical administration, the thrombolytic agent and/or the substance P receptor antagonist may be in the form of a solution, spray, lotion, cream (for example a non-ionic cream), gel, paste or ointment. Alternatively, the composition may be delivered via a liposome, nanosome, rivosome, or nutri-diffuser vehicle.

It will be appreciated that other forms of administration of agents are also contemplated, including the use of a nucleic acid encoding a polypeptide for delivering of such agents.

### DESCRIPTION OF SPECIFIC EMBODIMENTS

Reference will now be made to experiments that embody the above general principles of the present invention. However, it is to be understood that the following description is not to limit the generality of the above description.

### Example 1

Administration of N-acetyl-L-tryptophan Closes the Blood Brain Barrier Following Injury

A number of commercially synthesised substance P (NK1) receptor antagonists are available from standard scientific chemical suppliers.

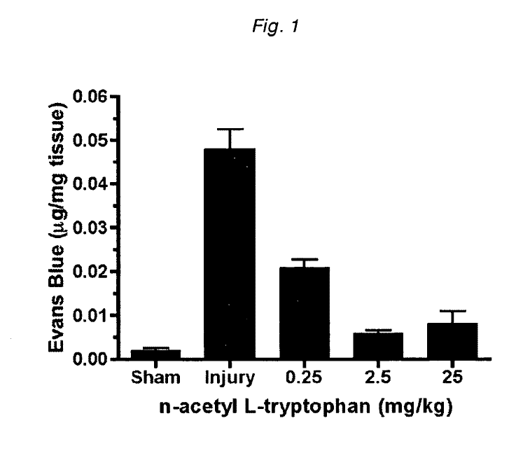

We chose to use the compound N-acetyl-L-tryptophan (NAT). Administration of N-acetyl-L-tryptophan was found to close the blood brain barrier following injury, as evidenced by a reduced leakage of Evan's blue dye into the brain tissue. This closure of the blood-brain barrier occurred in a dose-dependent manner, as shown in FIG. 1.

### Example 2

Administration of N-acetyl-L-tryptophan Prevents the Increased Permeability of the Blood Brain Barrier Induced by Injection of tPA

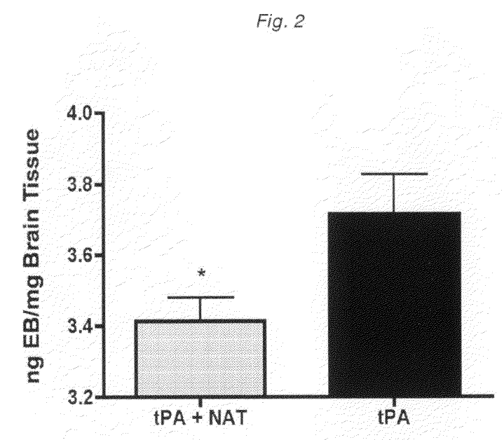

Administration of N-acetyl-L-tryptophan (NAT) at a dose of 10⁻⁵ mol/kg also prevented the increased permeability of the blood brain barrier induced by injection of tPA (Actilyse) alone to a naïve (uninjured) animal (FIG. 2), supporting the concept that NK1 antagonists are a useful adjunctive therapy to prevent tPA induced blood brain barrier opening.

### Example 3

Administration of N-acetyl-L-tryptophan Reduces Odemia Following Ischaemia/Reperfusion Injury

Having demonstrated that N-acetyl-L-tryptophan maintains barrier integrity both after injury and after administration of tPA, we sought to examine whether the administration of the NK1 antagonist would also reduce the oedema formation that usually accompanies blood brain barrier opening.

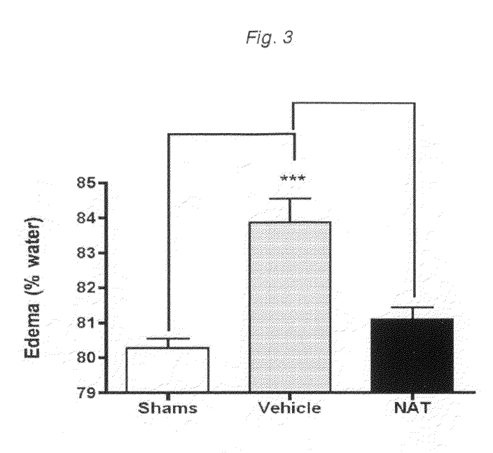

To this end, anaesthetised rats were subject to 2 hours reversible stroke using the intraluminal thread model of middle cerebral artery occlusion. At 2 hours, reperfusion was initiated by withdrawing the thread and following a further 2 hours, N-acetyl-L-tryptophan (10⁻⁵ mol/kg) was administered by intravenous injection. There was a significant increase in oedema at 24 h following ischaemia/reperfusion injury. This was significantly attenuated by the administration of N-acetyl-L-tryptophan (FIG. 3).

### Example 4

Administration of N-acetyl-L-tryptophan or L733,060 Improves Functional Outcome Following Ischaemia/Reperfusion Injury

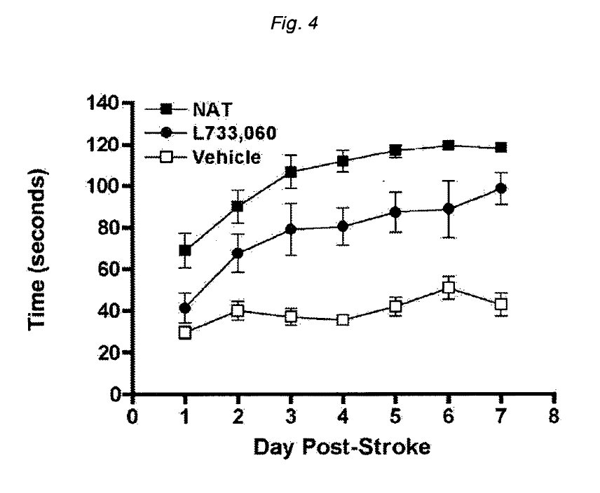

Anaesthetised rats were subject to 2 hours reversible stroke using the intraluminal thread model of middle cerebral artery occlusion. At 2 hours, reperfusion was initiated by withdrawing the thread and following a further 2 hours, N-acetyl-L-tryptophan (10⁻⁵ mol/kg) or L733,060 (1 mg/kg) was administered by intravenous injection. The administration of N-acetyl-L-tryptophan in these animals subject to ischaemia/reperfusion injury resulted in a profound improvement in functional outcome as assessed by the rotarod test of motor function over the ensuing 7 days (FIG. 4). Administration of the alternative substance P antagonist, L733,060, also improved functional outcome as assessed by the rotarod test of motor function over the ensuing 7 days (FIG. 4), demonstrating that the effects arc a class effect of substance P antagonists and not limited to N-acetyl-L-tryptophan.

### Example 5

Administration of N-acetyl-L-tryptophan Reduces Brain Inflammation Following Hypoxia and Hypotension

Another major aspect of reperfusion injury is the subsequent inflammatory response, as evidenced by increased expression of inflammatory mediators within the brain, as well as increased plasma levels of interleukin 6.

In order to induce the inflammatory response associated with reperfusion, anaesthetised rats were subject to 15 minutes of hypoxia (PAO₂ 30-40 mmHg) and hypotension (MABP 40 mmHg), before blood oxygen level and blood pressure were restored to normal levels.

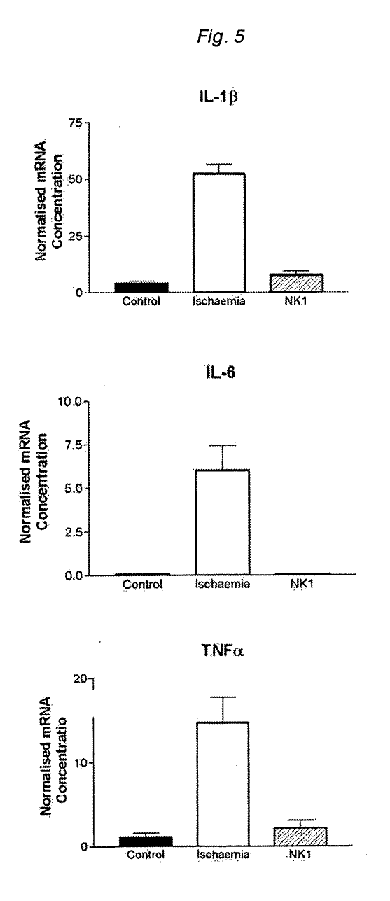

In order to examine the effects of a substance P antagonist on the inflammatory response, N-acetyl-L-tryptophan was administered intravenously at a dose of 10⁻⁵ mol/kg after hypoxia and hypotension. The expression of mRNA for the inflammatory cytokines IL-1β, IL-6 and TNFα in brain tissue was determined by reverse transcription followed by a polymerase chain reaction (RT-PCR). mRNA levels were normalised with respect to expression of rpL32. Brain tissue samples were collected at 6 hours after injury. Levels of expression of mRNA for the inflammatory cytokines were compared between non-ischaemic animals (control), ischaemic animals administered drug vehicle alone (ischaemia), and ischaemic animals administered a substance P receptor antagonist (NK1). mRNA expression was determined for IL-1β (FIG. 5a), IL-6 (FIG. 5b) and TNFα (FIG. 5c).

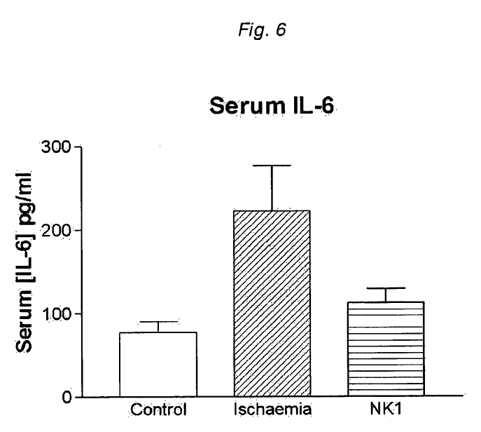

In addition, serum interleukin 6 levels were also determined. Blood samples were collected at 6 hours after the insult. Changes in serum IL-6 levels were quantified using an enzyme-linked immunosorbent assay (ELISA). Serum levels of IL-6 were compared between animals that were not subject to ischaemia (control), animals subject to ischaemia followed by administration of drug vehicle alone (ischaemia), and animals subject to ischaemia followed by administration of a substance P receptor antagonist (NK1) (FIG. 6).

Animals subject to ischaemia followed by reperfusion exhibited a marked inflammatory response, with increases in the expression of key inflammatory cytokines (IL-1β, IL-6 and TNFα) being observed. Animals treated with N-acetyl-L-tryptophan after injury did not exhibit this inflammatory response, with no significant differences in the expression of IL-1β, IL-6 and TNFα being observed with respect to non-ischaemic animals. Thus, N-acetyl-L-tryptophan administered after ischaemia, by inhibiting the neurogenic component of inflammation, was able to prevent the inflammatory response that is normally associated with reperfusion. Also, as seen previously in patients with brain ischaemia, increased serum IL-6 levels were observed following a period of hypoxia and hypotension. However in animals treated with N-acetyl-L-tryptophan after ischaemia, this response was not observed, indicating a suppression of the inflammatory response. A rise in serum IL-6 is normally taken as an indicator of poor patient prognosis.

Finally, it will be appreciated that various modifications and variations of the described methods and compositions of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are apparent to those skilled in the art are intended to be within the scope of the present invention.

1. A method of preventing and/or reducing reperfusion injury to a tissue, organ, or organ system in a subject, the method including administering to the subject an effective amount of a substance P receptor antagonist.

2. A method according to claim 1, wherein the reperfusion injury is due to administration to the subject of a thrombolytic agent to improve blood flow to the tissue, organ or organ system.

3. A method according to claim 2, wherein the thrombolytic agent is a tissue plasminogen activator.

4. A method according to claim 2 or 3, wherein the substance P receptor antagonist is a NK1 receptor antagonist, a NK2 receptor antagonist, or a NK3 receptor antagonist.

5. A method according to claim 4, wherein the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138, L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577, or a derivative, a variant, an analogue, a pharmaceutically acceptable salt, a tautomer or a pro-drug thereof.

6. A method according to claim 4, wherein the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, a tautomer or a pro-drug thereof.

7. A method according to claim 4, wherein the NK3 receptor antagonist is selected from one or more of the group consisting of SR-143,801, R820, R486, SB222200, L758,298 and NKP608, or a derivative, a variant, an analogue, a pharmaceutically acceptable salt, a tautomer or a pro-drug thereof.

8. A method according to any one of claims 1 to 7, wherein the organ system is the central nervous system.

9. A method according to any one of claims 1 to 8, wherein the organ is the brain.

10. A method according to claim 9, wherein the reperfusion injury to the brain includes injury due to an autoimmune response.

11. A method according to claim 9 or 10, wherein the reperfusion injury is due to reperfusion of all or part of the brain following a stroke.

12. Use of a substance P receptor antagonist in the preparation of a medicament for preventing and/or reducing reperfusion injury.

13. Use according to claim 12, wherein the reperfusion injury is due to administration of a thrombolytic agent.

14. Use according to claim 12 or 13, wherein the reperfusion injury occurs in the brain.

15. Use of a substance P receptor antagonist and a thrombolytic agent in the preparation of a medicament for preventing and/or treating a thrombo-embolic occlusion.

16. Use of a substance P receptor antagonist and a thrombolytic agent in the preparation of a medicament for preventing and/or treating stroke.

17. A combination product including the following components:
a substance P receptor antagonist; and
a thrombolytic agent;
wherein the components are provided in a form for separate administration to a subject, or in a form for co-administration to a subject.

18. A combination product according to claim 17 wherein the thrombolytic agent is a tissue plasminogen activator.

19. A combination product according to claim 17 or 18, wherein the substance P receptor antagonist is a NK1 receptor antagonist, a NK2 receptor antagonist, or a NK3 receptor antagonist.

20. A combination product accoriding to claim 19, wherein the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138, L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577, or a derivative, a variant, an analogue, a pharmaceutically acceptable salt, a tautomer or a pro-drug thereof.

21. A combination product according to claim 19, wherein the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, a tautomer or a pro-drug thereof.

22. A combination product according to claim 19, wherein the NK3 receptor antagonist is selected from one or more of the group consisting of SR-143,801, R820, R486, SB222200, L758,298 and NKP608, or a derivative, a variant, an analogue, a pharmaceutically acceptable salt, a tautomer or a pro-drug thereof.

23. A combination product according to any one of claims 17 to 22, wherein the subject is suffering from, or susceptible to, a thrombo-embolic occlusion.

24. A combination product according to any one of claims 17 to 23, wherein the subject is suffering from, or susceptible to, a stroke.

25. A pharmaceutical composition including an effective amount of a substance P receptor antagonist and a thrombolytic agent.

26. A pharmaceutical composition according to claim 25, wherein the thrombolytic agent is tissue plasminogen activator.

27. A pharmaceutical composition according to claim 25 or 26, wherein the substance P receptor antagonist is a NK1 receptor antagonist, a NK2 receptor antagonist, or a NK3 receptor antagonist.

28. A pharmaceutical composition according to claim 27, wherein the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138, L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577, or a derivative, a variant, an analogue, a pharmaceutically acceptable salt, a tautomer or a pro-drug thereof.

29. A pharmaceutical composition according to claim 27, wherein the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, a tautomer or a pro-drug thereof.

30. A pharmaceutical composition according to claim 27, wherein the NK3 receptor antagonist is selected from one or more of the group consisting of SR-143,801, R820, R486, SB222200, L758,298 and NKP608, or a derivative, a variant, an analogue, a pharmaceutically acceptable salt, a tautomer or a pro-drug thereof.

31. A method of preventing and/or reducing oedema to a tissue, organ or organ system in a subject due to reperfusion of the tissue, organ or organ system, the method including administering to the subject an effective amount of a substance P receptor antagonist.

32. A method according to claim 31, wherein the oedema is due to administration of a thrombolytic agent to improve blood flow to the tissue, organ or organ system.

33. A method according to claim 31 or 32, wherein the oedema is a cerebral oedema.

34. A method according to claim 33, wherein the cerebral oedema is due to administration of the thrombolytic agent to the subject to treat a stroke.

35. Use of a substance P receptor antagonist in the preparation of a medicament for preventing and/or reducing oedema due to reperfusion of a tissue, organ or organ system.

36. Use according to claim 35, wherein the reperfusion is due to administration of a thrombolytic agent to improve blood flow to the tissue, organ or organ system.

37. Use according to claim 35 or 36, wherein the oedema is a cerebral oedema.

38. A method of preventing and/or reducing an inflammatory response in a tissue, organ or organ system in a subject due to reperfusion of the tissue, organ or organ system, the method including delivering to the tissue, organ or organ system an effective amount of a substance P receptor antagonist.

39. A method according to claim 38, wherein the reperfusion is due to administration of a thrombolytic agent to improve blood flow to the tissue, organ or organ system.

40. A method according to claim 38 or 39, wherein the organ is the brain.

41. A method according to claim 40, wherein the inflammatory response is due to administration of the thrombolytic agent to treat a stroke.

42. Use of a substance P receptor antagonist in the preparation of a medicament for preventing and/or reducing an inflammatory response in a tissue, organ or organ system due to reperfusion of the tissue, organ or organ system.

43. Use according to claim 42, wherein the reperfusion is due to administration of a thrombolytic agent to improve blood flow to the tissue, organ or organ system.

44. Use according to claim 42 or 43, wherein the inflammatory response occurs in the brain.

45. A method of treating a thrombo-embolic occlusion in a subject, the method including administering to the subject an effective amount of a substance P receptor antagonist and a thrombolytic agent.

46. A method according to claim 45, wherein the thrombolytic agent is a tissue plasminogen activator.

47. A method according to claim 45 or 46, wherein the substance P receptor antagonist is a NK1 receptor antagonist, a NK2 receptor antagonist, or a NK3 receptor antagonist.

48. A method according to claim 47, wherein the NK1 receptor antagonist is selected from one or more of the group consisting of CGP49823, CP-96,345, CP99,994, CP-122,721, FK88, GR203040, GR205171, GR82334, GR94800, HSP-117, L-703,606 oxalate, L-732,138, L-733060, L-742,694, L-745,030, L-668,169, LY-303241, LY-303870, LY306740, MEN-11149, MK-869, PD-154075, R-544, RP-67580, RPR100893, Sendide, Spantide II, Spantide III, SR140333, WIN-41,7098, WIN-62,577, or a derivative, a variant, an analogue, a pharmaceutically acceptable salt, a tautomer or a pro-drug thereof.

49. A method according to claim 47, wherein the NK2 receptor antagonist is selected from one or more of the group consisting of SR-48968, L-659877, GR103537, MGN-10627, SR144190 and GR94800, or a derivative, a variant, an analogue, a pharmaceutically acceptable salt, a tautomer or a pro-drug thereof.

50. A method according to claim 47, wherein the NK3 receptor antagonist is selected from one or more of the group consisting of SR-143,801, R820, R486, SB222200, L758,298 and NKP608, or a derivative, a variant, an analogue, a pharmaceutically acceptable salt, a tautomer or a pro-drug thereof.

51. A method according to any one of claims 45 to 50, wherein the thrombo-embolic occlusion is a stroke, a pulmonary occlusion, or a coronary artery occlusion.

52. A method of treating an thrombo-embolic stroke in a subject, the method including administering to the subject an effective amount of a substance P receptor antagonist and a thrombolytic agent.

53. Use of a substance P receptor antagonist and a thrombolytic agent in the preparation of a medicament for treating a thrombo-embolic stroke.

54. A method of improving the prognosis or outcome of a subject suffering from a thrombo-embolic occlusion, the method including administering to the subject an effective amount of a substance P receptor antagonist and a thrombolytic agent.

55. A method according to claim 54, wherein the thrombo-embolic occlusion is a stroke.

56. A method according to claim 55, wherein the improvement in prognosis or outcome is an improvement in the motor and/or cognitive prognosis or outcome.
